(19) United States
(12) Patent Application Publication
(10) Pub. No.: US 2018/0087718 A1
(43) Pub. Date: Mar. 29, 2018

(54) DISPOSABLE SINGLE-USE CARTRIDGE FOR HOLDING COMPRESSED MEDICAL GAS

(71) Applicants: Frank Levy, Fort Myers, FL (US); Kimberley Levy, Fort Myers, FL (US)

(72) Inventors: Frank Levy, Fort Myers, FL (US); Kimberley Levy, Fort Myers, FL (US)

(21) Appl. No.: 15/808,965

(22) Filed: Nov. 10, 2017

Related U.S. Application Data
(63) Continuation-in-part of application No. 15/441,771, filed on Feb. 24, 2017.
Application No. 15/441,771 is a continuation of application No. 13/569,444, filed on Aug. 8, 2012, now Pat. No. 9,651,197.
Application No. 13/569,444 is a continuation-in-part of application No. 13/068,680, filed on May 17, 2011, now Pat. No. 8,876,749.
Application No. 13/068,680 is a continuation-in-part of application No. 12/652,845, filed on Jan. 6, 2010, now abandoned.
Application No. 12/652,845 is a continuation-in-part of application No. 12/210,368, filed on Sep. 15, 2008, now abandoned.
Application No. 12/210,368 is a continuation-in-part of application No. 11/945,674, filed on Nov. 27, 2007, now Pat. No. 7,543,760.
(60) Provisional application No. 60/867,323, filed on Nov. 27, 2006.

(51) Int. Cl. F17C 13/06; F17C 5/06; A01N 25/00; A61L 31/02

(52) U.S. Cl. CPC F17C 13/06; F17C 2201/058; F17C 5/06; F17C 2201/0104; A61K 9/124; A01N 25/00; F17C 2221/011; F17C 2221/013; A61L 31/022; F17C 2270/02

(57) ABSTRACT

A single-use cartridge accommodating a medical gas includes an elongate, generally cylindrical body having an interior chamber accommodating a compressed and sterile medical grade gas therein. The body is inwardly tapered proximate one end thereof and is joined to a reduced diameter and generally cylindrical leading end portion. The leading end portion including an opening communicably connected to the interior chamber of the cartridge body. The opening is selectively opened to allow for opening of the cartridge such that the compressed medical gas contained within the interior chamber of the cartridge is deliverable through the opening for use in a medical procedure. The cylindrical body and the end portion include a triple washed construction for holding the medical grade gas within the interior chamber of the cylindrical body under pressure.

### CROSS REFERENCE TO RELATED APPLICATIONS

This application is a continuation in part of U.S. patent application Ser. No. 15/441,771, filed Feb. 24, 2017, which is currently pending, which is a continuation of U.S. patent application Ser. No. 13/569,444 filed Aug. 8, 2012, which is now U.S. Pat. No. 9,651,197, which is a continuation-in-part of U.S. patent application Ser. No. 13/068,680 filed May 17, 2011, which is now U.S. Pat. No. 8,876,749, which is a continuation in part of U.S. patent application Ser. No. 12/652,845 filed Jan. 6, 2010, now abandoned, which is a continuation in part of U.S. patent application Ser. No. 12/210,368 filed Sep. 15, 2008, now abandoned, which is a continuation in part of U.S. patent application Ser. No. 11/945,674 filed Nov. 27, 2007, now U.S. Pat. No. 7,543,760, which claims the benefit of U.S. Provisional Patent Application Ser. No. 60/867,323 filed Nov. 27, 2006, the disclosures of which are incorporated herein by reference in their entirety.

### BACKGROUND OF THE INVENTION

### 1. Field of the Invention

This invention relates to a disposable single-use cartridge for accommodating compressed medical gases such as carbon dioxide, oxygen, nitrous oxide and the like.

### 2. Description of the Related Art

Large pressurized tanks conventionally contain compressed gases for use in medical treatments and procedures. These tanks are usually heavy, bulky and quite inconvenient to transport. They can also present a dangerous risk of explosion. Refilling such tanks also tends to be time consuming and logistically inconvenient. In addition, pressurized full-sized tanks are also apt to harbor mold, debris and rust within the tank. This can seriously jeopardize the quality and integrity of the accommodated gas. Contaminants are highly undesirable in medical applications where the gas should exhibit a high degree of purity.

### SUMMARY OF THE INVENTION

It is therefore an object of the present invention to provide a compact disposable single-use cartridge for conveniently containing various types of medical gas in a compressed condition such that the gas is readily and conveniently available for use in assorted medical applications.

It is a further object of this invention to provide a single-use cartridge for compressed medical gas that is compact, lightweight and easy to transport, use and replace for medical procedures.

It is a further object of this invention to provide a single-use cartridge for compressed medical gas that is extremely safe to use and which greatly reduces the risks associated with potential projectile events as a result of misuse of the cartridge.

It is a further object of this invention to provide a disposable single-use cartridge for compressed medical gas that does not require tedious and time consuming refilling.

It is a further object of this invention to provide a single-use cartridge for compressed medical gas, which eliminates or at least greatly reduces the formation of mold and the collection of rust and debris therein such that the gas is maintained in a sterile and extremely pure condition for medical use.

This invention features a disposable single-use cartridge for accommodating a compressed medical gas. The medical gas may include carbon dioxide, oxygen, nitrous oxide or any other gas that must be stored in a pressurized condition for medical use. The single-use cartridge includes an elongate, generally cylindrical body that is tapered toward and joined to a reduced diameter and substantially cylindrical end portion. The end portion includes an opening communicably connected to an inferior chamber of the cartridge body. The opening is sealed by a pierceable tip. The tip is selectively punctured to open the cartridge such that compressed medical gas contained within the interior chamber of the cartridge body may be delivered through the tip for use in a medical procedure or application.

In a preferred embodiment, the cartridge body and reduced end portion include a triple-washed canister for holding a medical grade gas under pressure. The canister may weigh between 4 grams to 50 grams and is preferably about 16 grams or 25 grams (that is, the canister includes the specified number of grams of gas in a liquid/gas equilibrium).

The reduced diameter portion of the canister may be encircled by a thread for attaching the single-use cartridge to a medical apparatus requiring compressed medical gas of the type contained by the single-use cartridge. The optimum thread size is approximately ⅜″, although it can vary from about ¼″ to 1″.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a side elevational view of a preferred disposable single-use cartridge for accommodating compressed medical gas in accordance with this invention.

FIGS. 2A and 2B are cross sectional side views of a preferred disposable single-use cartridge in opened and closed orientation in accordance with an alternate embodiment.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

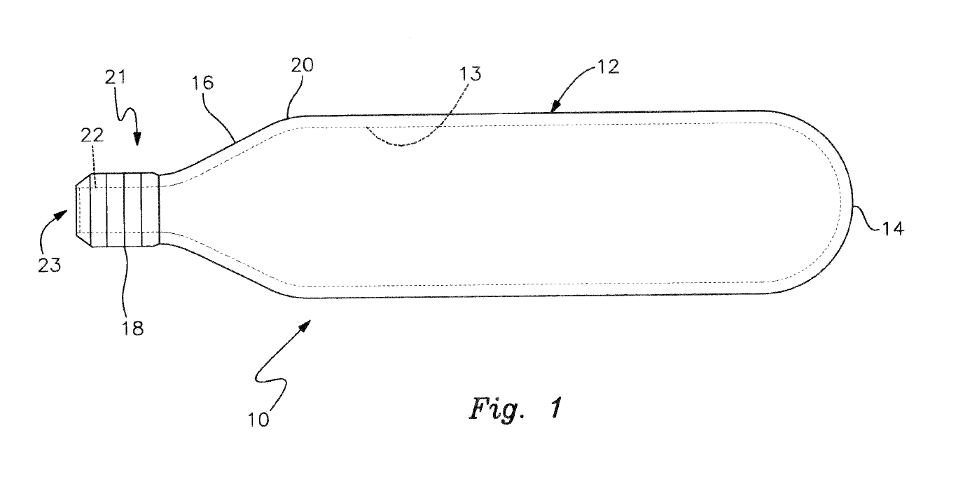

There is shown in FIG. 1 a disposable single-use aluminum, titanium, or steel cartridge 10 for accommodating a compressed medical gas. It should be understood that single-use cartridge 10 is suitable for holding a wide variety of medical gases including, but not limited to, carbon dioxide, oxygen and nitrous oxide. Single-use cartridge 10 is capable of holding virtually any type of medical gas in a pressurized condition for use in various types of medical procedures. The type of gas and the type of medical applications involved are not limitations of this invention.

Single-use cartridge 10 includes a generally cylindrical canister body 12 having an elongate shape with a rounded bottom 14. The canister body encloses an interior chamber 13 for holding a compressed medical gas. The body includes a tapered neck portion 16 that is joined to and terminates in a reduced diameter cylindrical end portion 18. Body 12, tapered portion 16 and reduced diameter portion 18 thereby define a canister 20 that is preferably composed of aluminum, titanium, steel, or other available material that may be properly sterilized for use in conjunction with medical grade gases and in the performance of the medical procedures.

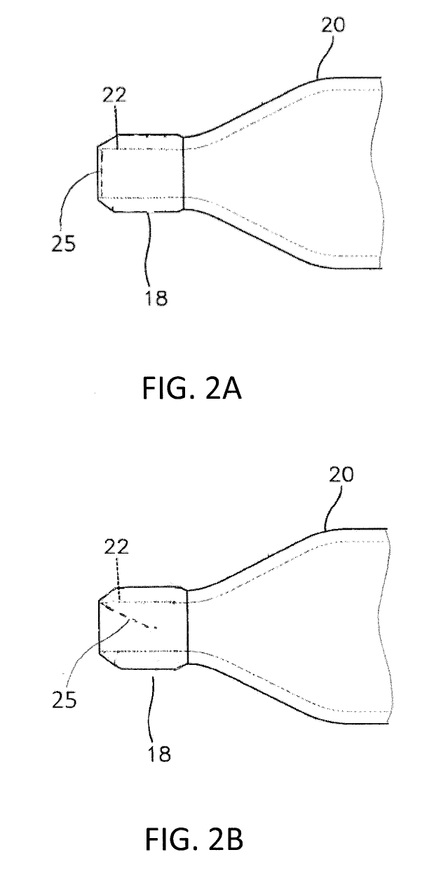

The end portion is preferably encircled by a thread 21 that allows canister 20 to be screwed into a complementary threaded opening of a piece of medical equipment with which the canister is engaged. The optimum thread size is ⅜″ although it can vary from ¼″ to 1″ within the scope of this invention. In accordance with an alternate embodiment, as disclosed with reference to FIGS. 2A and 2B, the end portion 18 may be constructed without threading. In accordance with such an embodiment, the device making use of the present single-use cartridge would require a holding member to secure the single-use cartridge in place as the compressed gas is released therefrom for use in conjunction with a medical procedure.

In radiological applications, canister 20 may be used to hold carbon dioxide in chamber 13 and the threaded reduced diameter end portion 18 may be engaged with the portable medical gas delivery system produced under the trademark CO2MMANDER® as disclosed in pending U.S. patent application Ser. Nos. 13/068,880 and 12/652,845, the disclosures of which are incorporated herein by reference. Although the cartridge is particularly convenient and effective for use with this system, it may be used in a wide variety of other medical applications and for holding various other types of compressed medical gases.

Reduced diameter portion 18 includes an opening 22 in communication with interior chamber 13. The distal end of reduced diameter portion 18 of canister 20 carries a pierceable tip 23 that seals opening 22 after pressurized medical gas has been introduced into the interior of canister 12. Tip 23 may comprise a fairly thin and flexible foil that is securely sealed over opening 22 in end portion 18. Tip 23 is readily punctured in a conventional manner to open canister 12 so that compressed gas within the canister is made available for delivering through opening 22 to a desired medical application or use.

In accordance with an alternate embodiment, and with reference to FIGS. 2A and 2B, the distal end of reduced diameter portion 18 of canister 20 carries a selectively sealable member in the form resiliently biased flap member 25 that seals opening 22 after pressurized medical gas has been introduced into the interior of canister 20, but allows for pressurized gas with canister 20 to be released through opening 22 in reduced diameter portion 18 when flap member 25 is contacted and forced to move from opening 22 at the distal end of reduced diameter portion 18. In practice, flap member 25 is forced rearwardly by a medical device secured to single-use cartridge 10 in a conventional manner such that opening 22 is opened to allow compressed gas within canister 20 to be made available for delivery through opening 22 to a desired medical application or use. In accordance with such an embodiment, flap member 25 is spring loaded and will reseal opening 22 in the event single-use cartridge 10 is removed from the medical device before the compressed gas is fully exhausted therefrom.

While a selectively sealable member in the form of a flap construction is disclosed above as a mechanism for allowing the selective release of the gas contained with the canister and the resealing of the opening when cartridge is removed from the portable medical gas delivery system, it is appreciated other mechanisms may be employed, for example, ball valves, check valves, butterfly valves, and air compressor coupling mechanisms.

Canister 12 is composed and constructed to provide significant advantages. In particular, the canister has a relatively compact size and configuration and is extremely lightweight especially when compared with standard full-sized tanks conventionally used to contain compressed medical gases. In particular, the preferred weight of canister 12 is 16 grams or 25 grams, although it can range from 4 grams to 50 grams (that is, the canister includes the specified number of grams of gas in a liquid/gas equilibrium). The canister employs a triple-washed aluminum, titanium or steel construction, which maintains the sterility and purity of gases contained within the canister. In particular, the triple-washed canister resists the formation of mold and rust, as well as the collection of other types of debris within the interior chamber 13 of the canister. As a result, the integrity of the canister is maintained so that the contained gas is suitable for medical use wherein high levels of purity and sterility are indispensable.

The compact and relatively small size of the single-use cartridge allows the single-use cartridge to be transported and manipulated easily and conveniently. Replacement and disposal of the single-use cartridge are facilitated. Bulky, heavy and cumbersome tanks are avoided.

The compact single-use cartridge is disposable and therefore does not have to be refilled or cleaned between uses. This reduces the time, logistic complexity and tedium required to transport and refill conventional tanks. It also helps to maintain the sterility and purity of the compressed medical gas contained within the single-use cartridge. The lightweight, disposable aluminum, titanium or steel single-use cartridge disclosed herein therefore provides for a number of significant advantages over the large bulky and cumbersome tanks used in the prior art.

From the foregoing it may be seen that the apparatus of this invention provides for a disposable aluminum, titanium or steel single-use cartridge for accommodating compressed medical gases such as carbon dioxide, oxygen, nitrous oxide and the like. While this detailed description has set forth particularly preferred embodiments of the apparatus of this invention, numerous modifications and variations of the structure of this invention, all within the scope of the invention, will readily occur to those skilled in the art. Accordingly, it is understood that this description is illustrative only of the principles of the invention and is not limitative thereof.

Although specific features of the invention are shown in some of the drawings and not others, this is for convenience only, as each feature may be combined with any and all of the other features in accordance with this invention.

1. A single-use cartridge accommodating a medical gas, the cartridge comprising:
an elongate, generally cylindrical body having an interior chamber accommodating a compressed and sterile medical grade gas therein, the body being inwardly tapered proximate one end thereof and joined to a reduced diameter and generally cylindrical leading end portion, the leading end portion including an opening communicably connected to the interior chamber of the cartridge body,
the opening being selectively opened to allow for opening of the cartridge such that the compressed medical gas contained within the interior chamber of the cartridge is deliverable through the opening for use in a medical procedure, wherein the cylindrical body and the end portion include a triple washed construction for holding the medical grade gas within the interior chamber of the cylindrical body under pressure.

2. The single-use cartridge according to claim 1, wherein the leading end portion includes threads.

3. The single-use cartridge according to claim 1, wherein the leading end portion does not include threads.

4. The single-use cartridge according to claim 1, wherein the opening includes a selectively sealable member.

5. The single-use cartridge according to claim 1, wherein the selectively sealable member is a resiliently biased flap.
